(19) United States
(12) Patent Application Publication
(10) Pub. No.: US 2015/0366877 A1
(43) Pub. Date: Dec. 24, 2015

(54) METHODS OF TREATMENT OF HUMAN CYTOMEGALOVIRUS INFECTION AND DISEASES WITH BROMODOMAIN INHIBITORS

(71) Applicant: Washington University, Saint Louis, MO (US)

(72) Inventors: Dong YU, Canton, MA (US); Yi-Chieh PERNG, Saint Louis, MO (US); Deborah Lenschow, Saint Louis, MO (US)

(21) Appl. No.: 14/839,425

(22) Filed: Aug. 28, 2015

Related U.S. Application Data
(63) Continuation of application No. PCT/US14/19701, filed on Feb. 28, 2014.
(60) Provisional application No. 61/770,886, filed on Feb. 28, 2013.

(51) Int. Cl. A61K 31/551

(52) U.S. Cl. CPC A61K 31/551

(57) ABSTRACT

Methods of inhibiting replication of human cytomegalovirus (HCMV) are disclosed. In various configurations, these methods comprise administering a therapeutically effective amount of a bromodomain inhibitor to a subject in need thereof. Bromodomain inhibitors including methyltriazolodiazepine-related compounds, 3,5-dimethylisoxazole-related compounds, 3-methyldihydroquinazolinone-related compounds, N-acetyl-2-methyltetrahydroquinoline-related compounds, quinazolone-related compounds, diazobenzene-related compounds, and triazolopyridazine-related compounds can be used to inhibit viral replication.

### CROSS-REFERENCE TO RELATED APPLICATIONS

This application claims the benefit of and priority to PCT/US14/19701 filed Feb. 28, 2014. PCT/US14/19701 claims the benefit of U.S. Provisional Patent Application 61/770,886 filed Feb. 28, 2013. Each of these applications is incorporated herein by reference each in its entirety.

### GOVERNMENT SUPPORT

This work received government support from National Institutes of Health under Grant No. NIH/NCI R01CA120768. The government may have certain rights in the invention.

### INTRODUCTION

HCMV infection is one of the most common sources of complications in cancer patients. Numerous compounds have been identified that inhibit the function of bromodomain-containing proteins. Some of these bromodomain inhibitors (sometimes referred to as BET bromodomain inhibitors), such as JQ1, have been applied to various disease, including cancers, inflammatory diseases, cardiovascular diseases, and male fertility (Anand, P., et al. 2013, Delmore, J. E., et al. 2011, Lockwood, W. W., et al., 2012; Ott, C. J., et al. 2012; Zuber, J., et al., 2011; Maxmen, A., et al. 2012; Filippakopoulos, P., et al., 2010; and Matzuk, M. M., et al., 2012). JQ1 and its derivatives have been in clinical trials for its anti-cancer application.

Palermo, R. D., et al., 2011, found that treating cells with JQ1 inhibits production of transcripts in Epstein-Barr virus (EBV). These authors also suggest the use of JQ1 as a potential anti-EBV agent. However, these transcripts are unique in EBV for its long-term latency/oncogenesis in B cells and are not conserved among herpesviruses. EBV and HCMV are different viruses; they affect different cell types, and have different disease manifestations.

PCT applications PCT/US2011/036701 and PCT/US2011/036647 of Bradner, J. E., et al, PCT/EP2010/066714 of Bamborough et al., PCT applications PCT/US2011/063173, PCT/US2012/036569, PCT/US2012/042825, and PCT/US2013/044444 of Albrecht, B. K., et al., PCT/EP2012/066600 of Schmees, N., et al., PCT/IB2012/054211 of Fish, P. V., et al., PCT/EP2010/066701 of Demont, E. H., et al., PCT/EP2010/061518 of Gosmini, R. L. M., et al., and US Patent Application US20120028912 A1 of Zhou, M. M, et al. do not disclose treatment of HCMV by administering bromodomain inhibitors. Furthermore, some viruses are believed to use BRD4 to anchor its viral DNA to a host chromosome. However, HCMV does not use BRD4 as an anchor; instead, it is believed to use its own 1E-1 protein for this purpose (Mücke, K., et al. 2014). Therefore, it was unknown whether bromodomain inhibitors can be used to inhibit HCMV infection.

PCT applications PCT/EP2010/066697 of Bailey, J., et al., PCT/EP2010/066695 of Chung, C., et al, PCT/CN2012/086357 of Wang, L., et al., and PCT/EP2010/066699 of Bouillot, A. M. J., et al. state that bromodomain inhibitors may be useful for treating inflammatory responses. However, treating an inflammatory response caused by a virus is different than treating the viral infection. These applications do not disclose use of bromodomain inhibitors to treat HCMV infection.

PCT application PCT/IB2013/000968 of McLure, K. G., et al. describes quinazolinone derivatives as bromodomain inhibitors and states that bromodomain inhibitors may modulate responses to viral infections including herpes, HPV, and HIV. McLure also states that the disclosed compositions may be employed to treat diseases or disorders caused by viral infections. However, treating disease symptoms caused by a viral infection is different than treating the viral infection itself. PCT/IB2013/000968 does not disclose examples supporting using the compositions disclosed in PCT/IB2013/000968 for treating beta-herpesviruses infections including HCMV.

There are no published disclosures that describe the use of bromodomain inhibitors including JQ1 or its derivatives to inhibit infection of human cytomegalovirus (HCMV).

### SUMMARY

The present inventors have shown that various bromodomain inhibitors can interfere with viral replication of a cytomegalovirus including a human cytomegalovirus (HCMV). Bromodomain inhibitors can thus be used therapeutically against cytomegalovirus infection.

In some embodiments, the present inventors disclose methods of inhibiting replication of human cytomegalovirus (HCMV) in a subject. In various configurations, these methods comprise administering a therapeutically effective amount of a bromodomain inhibitor to a subject in need thereof.

In some embodiments, the present inventors disclose methods of treating a human cytomegalovirus (HCMV) infection in a subject. In various configurations, these methods comprise administering a therapeutically effective amount of a bromodomain inhibitor to a subject in need thereof.

In some embodiments, the present inventors disclose use of a bromodomain inhibitor for the treatment of human cytomegalovirus (HCMV) infection.

In some embodiments, the present inventors disclose methods of inhibiting human cytomegalovirus (HCMV) replication in vitro. In various configurations, these methods comprise providing a culture comprising a host cell infected with HCMV, and contacting the host cell with a bromodomain inhibitor.

In various configurations, bromodomain inhibitors, including inhibitors against the bromo and extra terminal (BET) family of bromodomains can be used with the disclosed methods.

Bromodomain inhibitors of the present teachings include, in various configurations, methyltriazolodiazepine-related compounds, 3,5-dimethylisoxazole-related compounds, 3-methyldihydroquinazolinone-related compounds, N-acetyl-2-methyltetrahydroquinoline-related compounds, quinazolone-related compounds, diazobenzene-related compounds, triazolopyridazine-related compounds, and pyrrolopyridinone-related compounds.

A methyltriazolodiazepine-related compound of the present teachings can be, without limitation, (+)JQ-1 (TEN-10)(4-(4-chlorophenyl)-2,3,9-trimethyl-1,1-dimethylethyl ester-6H-thieno[3,2-f][1,2,4]triazolo[4,3-}{1,4}diazepine-6S-acetic acid), I-BET 762 (GSK525762A) (2-[(4S)-6-(4-chlorophenyl)-8-methoxy-1-methyl-4H-[1,2,4]triazolo[4,3-a][1,4]benzodiazepine-4-yl]-N-ethylacetamide), OTX-015 ((S)-2-[4-(4-chlorophenyl)-2,3,9-trimethyl-6H-thieno [3,2-f][1,2,4]triazolo-[4,3-a][1,4]diazepin-6-yl]-N-(4-hydroxyphenyl)acetamide), CPI-203 ((S)-2-(4-(4-chlorophenyl)-2,3,9-trimethyl-6H-thieno [3,2-f][1,2,4]triazolo[4,3-a][1,4]diazepin-6-yl)acetamide), a 6-spiro-substituted triazolodiazepine such as (1R,2R)-4′-(4-Chlorophenyl)-N-ethyl-2′,3,9-trimethylspiro-[cyclopropane-1,6′-thieno [3,2-f][1,2,4]triazolo[4,3-a][1,4]diazepine]-2-carboxamide, a dihydrobenzodiazepine such as 4H-[1,2,4]triazolo[4,3-a][1,5]benzodiazepine,5,6-dihydro-1,4-dimethyl-8-(6-aminopyridin-&3-yl)-6-(4-chloro- phenyl), an isoxazoloazepine, a 6h-thieno[3,2-f][1,2,4]triazolo[4,3a][1,4]diazepine or MS-417 (Methyl 2-((6S)-4-(4-chlorophenyl)-2,3,9-trimethyl-6H-thieno[3,2-f][1,2,4]triazolo[4,3-a][1,4]diazepin-6-yl)acetate.

A 3,5-methylisoxazole-related compound of the present teachings can be, without limitation, I-BET 151 (GSK1210151A) (7-(3,5-Dimethyl-1,2-oxazol-4-yl)-8-methoxy-1-[(1R)-1-(2-pyridinyl)ethyl]-1,3-dihydro-2H-imidazo[4,5-c]quinolin-2-one).

A 3-methyldihydroquinazolinone-related compound of the present teachings can be, without limitation, PFI-1 (2-Methoxy-N-(3-methyl-2-oxo-1,2,3,4-tetrahydro-6-quinazolinyl)benzenesulfonamide).

An N-acetyl-2-methyltetrahydroquinoline-related compound of the present teachings can be, without limitation, I-BET 726 (GSK 1324726A)(4-(2S, 4R)-{-1-acetyl-4-[(4-chlorophenyl)amino]-2-methyl-1,2,3,4-tetrahydro-6-quinolinyl}benzoic acid.

A quinazolone-related compound of the present teachings can be, without limitation, RVX-208 (2-[4-(2-hydroxyethoxy)-3,5-dimethyl-phenyl]-5,7-dimethoxy-3H-quinazolin-4-one).

A diazobenzene related compound of the present teachings can be, without limitation, MS436 (2-[4-(2-hydroxyethoxy)-3,5-dimethyl-phenyl]-5,7-dimethoxy-3H-quinazolin-4-one.)

A triazolopyridazine-related compound of the present teachings can be, without limitation, a triazolopyridazine such as (S)-1-ethyl-3-(3-methyl-6-(methyl(1-phenylethyl) [1,2,4]triazolo[4,3-b]pyridazin-8-yl)urea, or bromosporine (N-[6-(3-methanesulfonamido-4-methylphenyl)-3-methyl-[1,2,4]triazolo[4,3-b]pyridazin-8-yl]carbamate).

A pyrrolopyridinone-related compound of the present teachings can be, without limitation, a pyrrolopyridinone such as N-[4(2,4-difluorophenoxy)-3-(6-methyl-7-oxo-6,7-dihydro-1H-pyrrolo[2,3-c]pyridine-4-yl)phenyl]ethanesulfonamide.

A bromodomain inhibitor of the present teachings can be, without limitation, a compound set forth in Table 1:

TABLE 1
Bromodomain Inhibitors

| Name | Structure | Source |
|---|---|---|
| (+)-JQI | 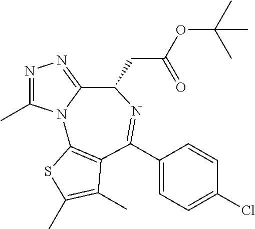 [SMILES: Cc1sc2c(c1C)C(c1ccc(Cl)cc1)=N[C@@H](CC(=O)OC(C)(C)C)c1nnc(C)n1-2] | Tensha Therapeutics |
| PF1-1 | 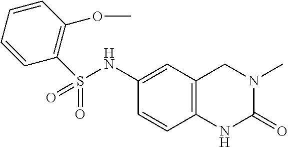 [SMILES: COc1ccccc1S(=O)(=O)Nc1ccc2c(c1)CN(C)C(=O)N2] | Pfizer |
| 1-BET 762 GSK525762A | 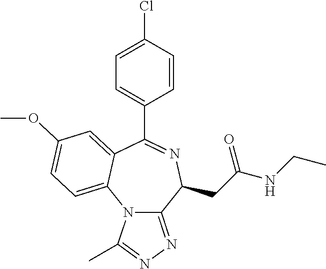 [SMILES: CCNC(=O)C[C@@H]1N=C(c2ccc(Cl)cc2)c2cc(OC)ccc2-n2c(C)nnc21] | GlaxoSmithKline |
| RVX-208 | 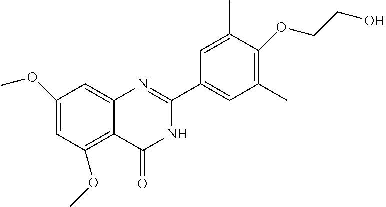 [SMILES: COc1cc(OC)c2c(=O)[nH]c(-c3cc(C)c(OCCO)c(C)c3)nc2c1] | Resverlogix |
| 1-BET 151 GSK1210151A | 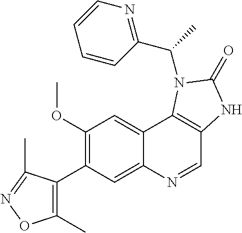 [SMILES: COc1cc2c(cc1-c1c(C)noc1C)ncc1[nH]c(=O)n([C@@H](C)c3ccccn3)c12] | GlaxoSmithKline |
| OTX-15 | 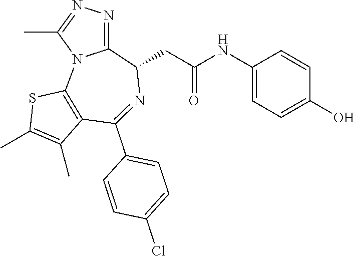 [SMILES: Cc1sc2c(c1C)C(c1ccc(Cl)cc1)=N[C@@H](CC(=O)Nc1ccc(O)cc1)c1nnc(C)n1-2] | Mitsubishi Tanabe/Oncoethix |
| CP1-203 | 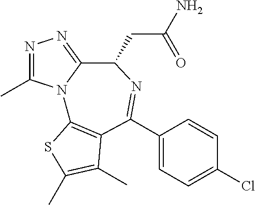 [SMILES: Cc1sc2c(c1C)C(c1ccc(Cl)cc1)=N[C@@H](CC(N)=O)c1nnc(C)n1-2] | Constellation Pharmaceuticals |
| bromosporine | 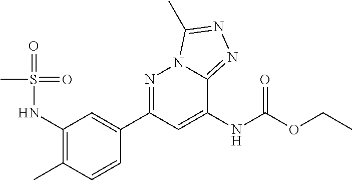 [SMILES: CCOC(=O)Nc1cc(-c2ccc(C)c(NS(C)(=O)=O)c2)nn2c(C)nnc12] | SGC |
| 1-BET 726 GSK 1324726A | 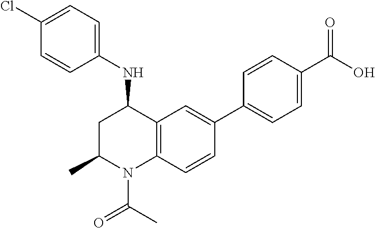 [SMILES: CC(=O)N1c2ccc(-c3ccc(C(=O)O)cc3)cc2[C@H](Nc2ccc(Cl)cc2)C[C@@H]1C] | GlaxoSmithKline |
| 6-Spiro-substituted triazolodiazepine | 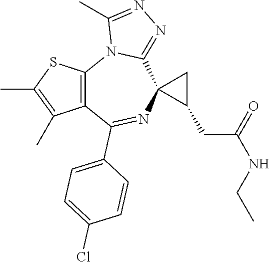 [SMILES: CCNC(=O)C[C@H]1C[C@]12N=C(c1ccc(Cl)cc1)c1c(sc(C)c1C)-n1c(C)nnc12] | Constellation Pharmaceuticals |
| dihydrobenzodiazepine | 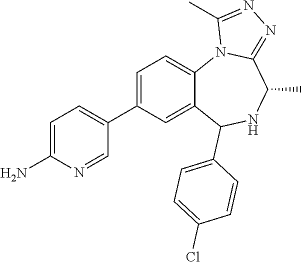 [SMILES: Cc1nnc2n1-c1ccc(-c3ccc(N)nc3)cc1C(c1ccc(Cl)cc1)N[C@H]2C] | Constellation Pharmaceuticals |
| isoxazoloazepine | 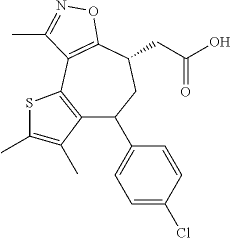 [SMILES: Cc1noc2c1-c1sc(C)c(C)c1C(c1ccc(Cl)cc1)C[C@H]2CC(=O)O] | Constellation Pharmaceuticals |
| 6h-thieno[3,2-f] [1,2,4]triazolo[4,3-a] [1,4]diazepine | 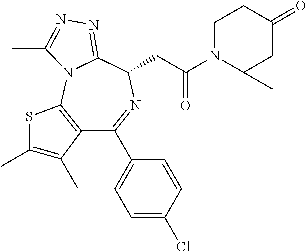 [SMILES: Cc1sc2c(c1C)C(c1ccc(Cl)cc1)=N[C@@H](CC(=O)N1CCC(=O)CC1C)c1nnc(C)n1-2] | Bayer Intellectual Property Gmbh |
| MS-417 | 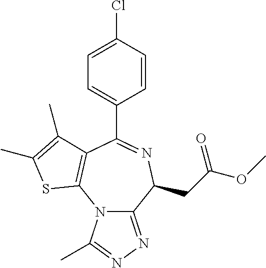 | Mount Sinai School of Medicine |
| 1-BET 726 GSK 1324726A | 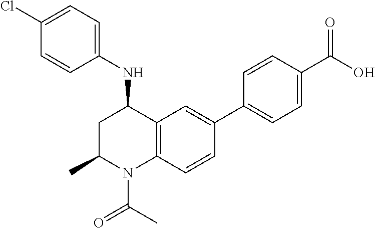 [SMILES: CC(=O)N1c2ccc(-c3ccc(C(=O)O)cc3)cc2[C@H](Nc2ccc(Cl)cc2)C[C@@H]1C] | GlaxoSmithKline |
| MS-436 | 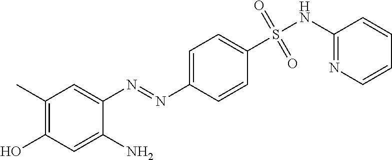 [SMILES: Cc1cc(/N=N/c2ccc(S(=O)(=O)Nc3ccccn3)cc2)c(N)cc1O] | Mount Sinai School of Medicine |
| triazolopyridazine | 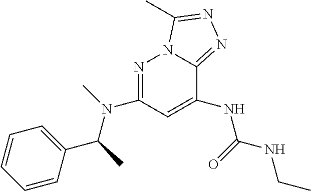 [SMILES: CCNC(=O)Nc1cc(N(C)[C@@H](C)c2ccccc2)nn2c(C)nnc12] | Constellation Pharmaceuticals |
| pyrrolopyridinone | 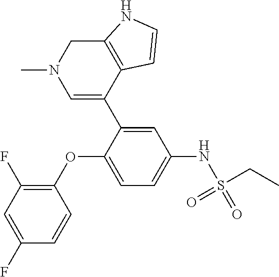 [SMILES: CCS(=O)(=O)Nc1ccc(Oc2ccc(F)cc2F)c(C2=CN(C)Cc3[nH]ccc32)c1] | Abbvie, Inc. |

A bromodomain inhibitor of the present teachings can be, without limitation, a compound set forth in Table 2:

TABLE 2
Bromodomain Inhibitors

| Chemical Type | Name | Inventors/Company |
|---|---|---|
| Methyltriazolodiazepines-related | JQ-1 (TEN-010) | Tensha therapeutics |
| | I-BET 762 | GlaxoSmithKline |
| | OTX-015 | Mitsubishi |
| | | Tanabe/Oncoethix |
| | CPI-203 | Constellation |
| | | Pharmaceuticals |
| | 6-Spiro-substituted | Constellation |
| | triazolodiazepines | Pharmaceuticals |
| | Dihydrobenzodiazepines | Constellation |
| | | Pharmaceuticals |
| | Isoxazoloazipines | Constellation |
| | | Pharmaceuticals |
| | 6h-thieno[3,2-f][1,2,4]triazolo[4,3- | Bayer Intellectual |
| | a][1,4]diazepines | Property Gmbh |
| | M5-417 | Mount Sinai School |
| | | of Medicine |
| 3,5-Dimethylisoxazoles-related | I-BET 151 | GlaxoSmithKline |
| 3-Methyldihydroquinazolines-related | PFI-1 | Pfizer |
| N-acetyl-2-methyltetrahydroquinolines- | I-BET 726 | GlaxoSmithKline |
| related | | |
| Quinazolone-related | RVX-208 | Resverlogix |
| Diazobenzene-related | MS436 | Mount Sinai School |
| | | of Medicine |
| Triazolopyridazines-related | Triazolopyridazines | Constellation |
| | | Pharmaceuticals |
| | Bromosporine | SGC |
| Pyrrolopyridinones-related | Pyrrolopyridinones | Abbvie, Inc. |

In some embodiments, a bromodomain inhibitor which can be used in methods of the present teachings can have a structure

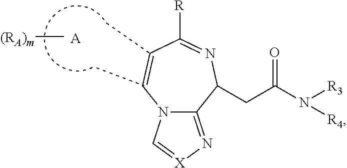

wherein X is N or CR₅; R₅ is H, alkyl, cycloalkyl, heterocycloalkyl, aryl, or heteroaryl, each of which is optionally substituted; R_{B} can be H alkyl, hydroxylalkyl, aminoalkyl, alkoxyalkyl, haloalkyl, hydroxy, alkoxy, or —COO—R₃, each of which is optionally substituted; ring A can be aryl or heteroaryl; each R_{A} can be independently alkyl, cycloalkyl, heterocycloalkyl, aryl, or heteroaryl, each of which is optionally substituted; or any two R_{A} together with the atoms to which each is attached, can form a fused aryl or heteroaryl group; R is alkyl, cycloalkyl, heterocycloalkyl, aryl, or heteroaryl, each of which is optionally substituted; each R₃ can be independently selected from the group consisting of: (i) H, aryl, substituted aryl, heteroaryl, or substituted heteroaryl; (ii) heterocycloalkyl or substituted heterocycloalkyl; (iii) —C₁-C₈ alkyl, —C₂-C₈ alkenyl or —C₂-C₈ alkynyl, each containing 0, 1, 2 or 3 heteroatoms selected from O, S, or N; —C₃-C₁₂ cycloalkyl, substituted —C₃-C₁₂ cycloalkyl, —C₃-C₁₂ cycloalkenyl, or substituted —C₃-C₁₂ cycloalkenyl, each of which may be optionally substituted; and (iv) NH₂, N═CR₄R₆; each R₄ ca be independently H, alkyl, alkyl, cycloalkyl, heterocycloalkyl, aryl, or heteroaryl, each of which is optionally substituted; or R₃ and R₄ can be taken together with the nitrogen atom to which they are attached to form a 4-10-membered ring; R₆ can be alkyl, alkenyl, cycloalkyl, cycloalkenyl, heterocycloalkyl, aryl, or heteroaryl, each of which is optionally substituted; or R₄ and R₆ are taken together with the carbon atom to which they are attached to form a 4-10-membered ring; m is 0, 1, 2, or 3; provided that: (a) if ring A is thienyl X is N, R is phenyl or substituted phenyl, R_{B} is methyl then R₃ and R₄ are not taken together with the nitrogen atom to which they are attached to form a morpholino ring; and (b) if ring A is thienyl, X is N, R is substituted phenyl, R₂ can be H, R_{B} is methyl, and one of R₃ and R₄ is H, then the other of R₃ and R₄ is not methyl, hydroxyethyl, alkoxy, phenyl, substituted phenyl, pyridyl or substituted pyridyl or a salt, solvate or hydrate thereof.

In some configurations, R can be aryl or heteroaryl, each of which can be optionally substituted.

In some configurations, R can be phenyl or pyridyl, each of which can be optionally substituted.

In some configurations, R can be p-Cl-phenyl, o-Cl-phenyl, m-Cl-phenyl, p-F-phenyl, o-F-phenyl, m-F-phenyl or pyridinyl.

In some configurations, R₃ can be H, NH₂, or N═CR₄R₆.

In some configurations, each R₄ can be independently H, alkyl, cycloalkyl, heterocycloalkyl, aryl or heteroaryl; each of which is optionally substituted.

In some configurations, R₆ can be alkyl alkenyl, cycloalkyl, cycloalkenyl, heterocycloalkyl, aryl or heteroaryl, each of which is optionally substituted.

The present teachings include pharmaceutical formulations for treatment of HCMV infection, and methods of administration of a pharmaceutical formulation for treatment of HCMV infection. Such pharmaceutical formulations can comprise a bromodomain inhibitor and an excipient. Administration can be by any administration route known to skilled artisans, such as, without limitation, injection, oral, or parenteral administration.

### BRIEF DESCRIPTION OF THE DRAWINGS

FIG. 1 illustrates that human cytomegalovirus (CMV) infected cells lose “cytomegaly” morphology and die upon JQ1 treatment. (A) Infected cells in phase-contrast or fluorescence microscopy at 72 hours post infection. (B) Infected cells in phase-contrast or fluorescence microscopy at 96 hours post infection.

FIG. 2 illustrates JQ1 inhibition of HCMV replication. (A) Number of viral progeny in media after 5 days post infection. (B) Number of viral progeny in media after 6 days post infection.

FIG. 3 illustrates IC₅₀ of JQ1 against HCMV replication using 4 and 3 parameter calculations.

FIG. 4 illustrates that JQ1 only modestly inhibits the accumulation of HCMV late proteins.

FIG. 5 illustrates transmission electron micrographs of human cytomegalovirus (HCMV)-infected fibroblasts.

FIG. 6 illustrates that representative examples of BET bromodomain inhibitors inhibit HCMV infection and spread.

FIG. 7 illustrates representative in vitro dose-responsive curves of BET bromodomain inhibitors for HCMV laboratory and clinical strains.

FIG. 8 illustrates representative in vitro dose-responsive curves of BET bromodomain inhibitors and current FDA-approved CMV antivirals.

FIG. 9 illustrates sensitivities of HCMV laboratory and clinical strains to BET bromodomain inhibitors determined by the release of viral particles (TCID₅₀ assay of culture supernatant).

FIG. 10 illustrates effect of the time of addition of current CMV anti-virals (Ganciclovir, Letermovir, or Cidofovir) or representative BET bromodomain inhibitors ((+)-JQ1, I-BET 762, or OTX-015) on HCMV replication.

FIG. 11 illustrates transmission electron micrographs of HCMV clinical strain-infected fibroblast in the presence or absence of representative bromodomain inhibitor (+)-JQ-1.

FIG. 12 illustrates representative bromodomain inhibitor (JQ-1) inhibits the transcription of genes involved in glutamine uptake and metabolism induced by HCMV infection.

### DETAILED DESCRIPTION

### Abbreviations

AC: cytoplasmic assembly compartments
BET: bromodomain and extra terminal
BRD: bromodomain
CMV: cytomegalovirus
Cyt: cytoplasm
DPI: days post infection
GFP: green fluorescent protein
GFPU: GFP units
EM: electronic microscopy
HCMV: Human cytomegalovirus
HFF: human foreskin fibroblasts
hpi: time post infection
IC: inhibitory concentration
MOI: multiplicity of infection
Nuc: nucleus
PBS: phosphate buffered saline
TCID: tissue culture infectious dose

### Methods

The methods and compositions described herein utilize laboratory techniques well known to skilled artisans, and can be found in laboratory manuals such as Sambrook, J., et al., Molecular Cloning: A Laboratory Manual, 3rd ed. Cold Spring Harbor Laboratory Press, Cold Spring Harbor, N.Y., 2001; Spector, D. L. et al. Cells: A laboratory Manual, Cold Spring Harbor Laboratory Press, Cold Spring Harbor, N.Y., 1998; Nagy, A., Manipulating the Mouse Embryo: A Laboratory Manual (Third Edition), Cold Spring Harbor, N.Y., 2003 and Harlow, E., Using Antibodies: A Laboratory Manual, Cold Spring Harbor Laboratory Press, Cold Spring Harbor, N.Y., 1999. Methods of administration of pharmaceuticals and dosage regimes, can be determined according to standard principles of pharmacology well known skilled artisans, using methods provided by standard reference texts such as Remington: the Science and Practice of Pharmacy (Alfonso R. Gennaro ed. 19th ed, 1995); Hardman, J. G., et al., Goodman & Gilman's The Pharmacological Basis of Therapeutics, Ninth Edition, McGraw-Hill, 1996; and Rowe, R. C., et al., Handbook of Pharmaceutical Excipients, Fourth Edition, Pharmaceutical Press, 2003. As used in the present description and the appended claims, the singular forms “a”, “an” and “the” are intended to include the plural forms as well, unless the context indicates otherwise.

### EXAMPLES

The present teachings including descriptions provided in the Examples that are not intended to limit the scope of any claim or aspect. Unless specifically presented in the past tense, an example can be a prophetic or an actual example. The following non-limiting examples are provided to further illustrate the present teachings. Those of skill in the art, in light of the present disclosure, will appreciate that many changes can be made in the specific embodiments that are disclosed and still obtain a like or similar result without departing from the spirit and scope of the present teachings.

### Example 1

This example demonstrates that HCMV cells lose “cytomegaly” morphology and die upon (+)-JQ-1 treatment.

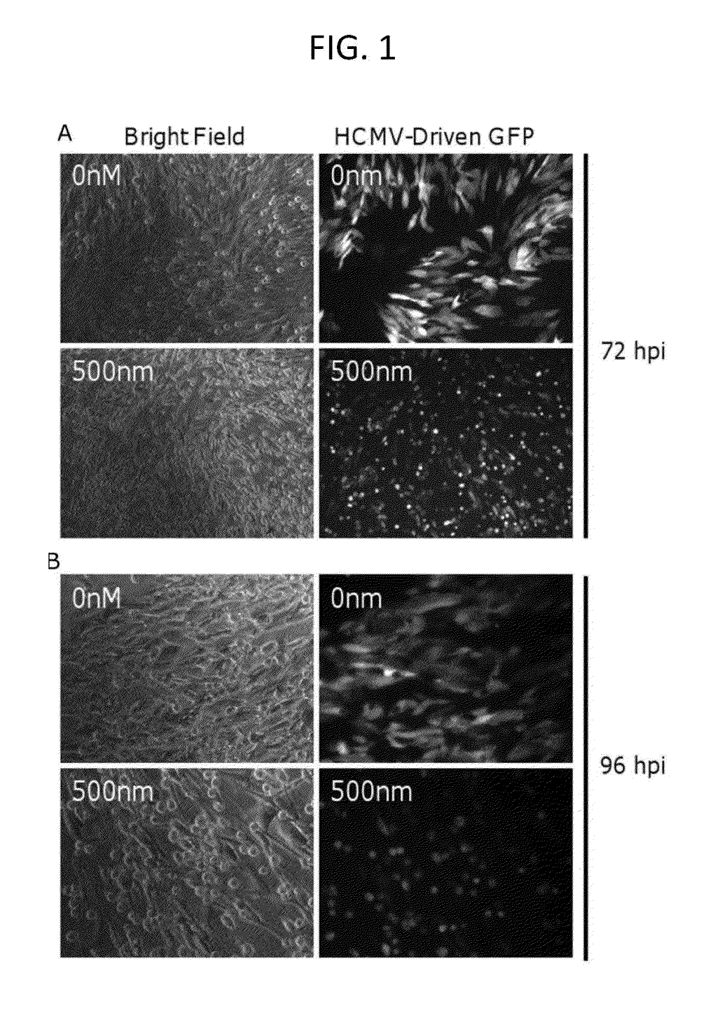

In these experiments, human foreskin fibroblasts (HFF) were infected with HCMV, strain AD169, at a multiplicity of infection (MOI) of 3 in the presence or absence of JQ1 (500 nm). Culture media was changed every 24 hours to maintain the concentration of JQ1. Infected cells were examined by phase-contrast or fluorescence microscopy at 72 or 96 hours post infection (hpi). “Cytomegalic” cells appear larger in size with a characteristic intranuclear, homogenous, eosinophilic inclusion which can occupy the entire nucleus of the cell. After 72 hours post-infection in the absence of JQ1, HFF cells displayed a “cytomegalic” morphology (FIG. 1A). While 72 hours after post-infection in the presence of JQ1, HFF cells lost the “cytomegalic” morphology and an accumulation of dead cells was present (FIG. 1A). After 96 hours post-infection in the absence of JQ1, HFF cells displayed a “cytomegalic” morphology (FIG. 1B). While 96 hours after post-infection in the presence of JQ1, HFF cells lost the “cytomegalic” morphology and a greater accumulation of floating dead cells were present as compared to 72 hours post-infection (FIG. 1B). These data demonstrate that HCMV infected cells lose “cytomegaly” morphology and die upon JQ1 treatment. Without being limited by theory, losing cytomegaly suggests that the lipogenesis of HCMV is disrupted.

### Example 2

This example demonstrates that representative BET bromodomain inhibitor JQ1 inhibits production of HCMV viral progeny.

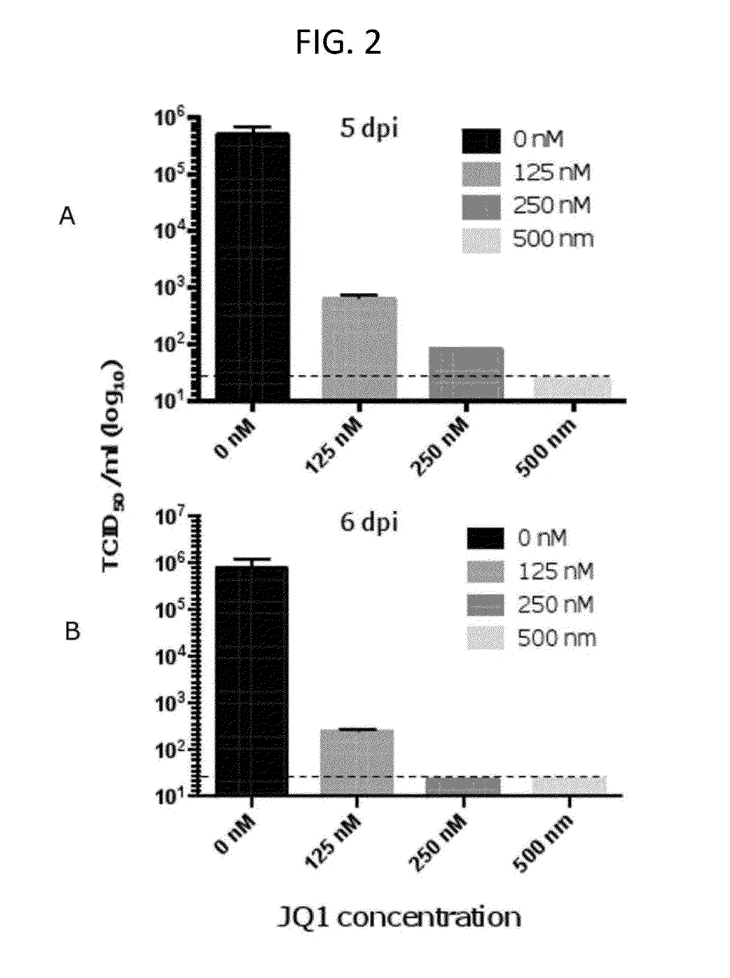

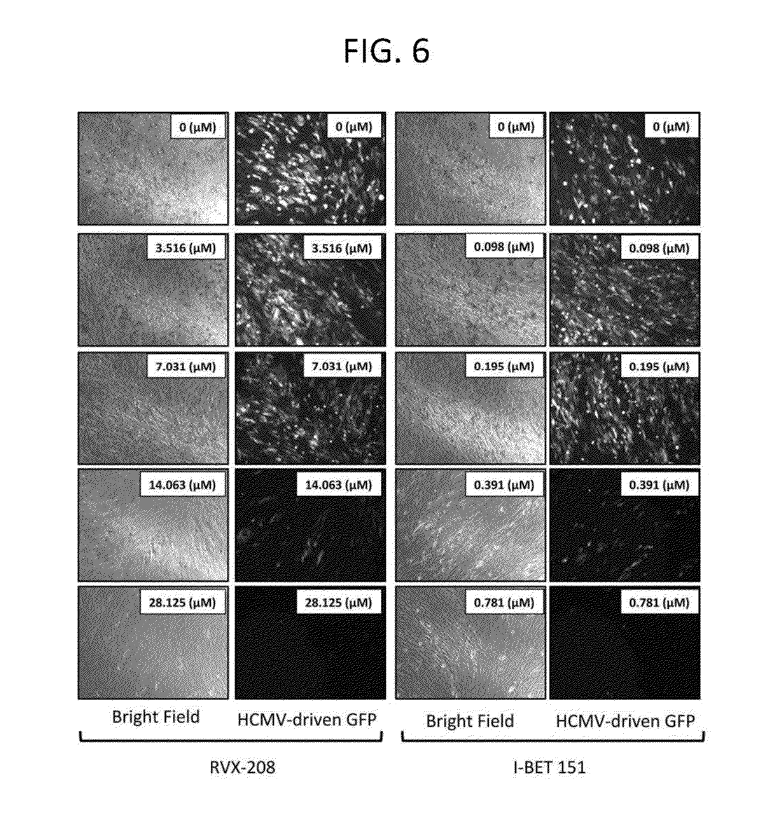

In this experiment, the inventors used TCID₅₀ assays to determine the amounts of infectious viral particle in culture supernatants release from HCMV-infected cells, HFFs were infected with HCMV, strain AD169, at an MOI of 3 in the presence of different concentrations of JQ1. Culture media was changed every 24 hours to maintain the concentration of JQ1. At 5 (FIG. 2A) and 6 (FIG. 2B) days post infection (DPI), infected culture media was collected and titers of viral progeny in media was determined by TCID₅₀ assay as described by Perng et al., 2011. The detection limit is indicated by the dashed line. (FIG. 2)

At 5 days post infection, 125 nM dose of JQ1 reduced the viral titer by approximately 1000 fold (FIG. 2A), increasing the concentration of JQ1 to 250 nM dose further reduced the viral titer and at 500 nM dose of JQ1 the viral titer was undetectable. At 6 days, post infection, 125 nM dose of JQ1 reduced the viral titer by greater than 1000 fold (FIG. 2B). The viral titer was undetectable at 250 nM and 500 nM doses of JQ1 after 6 days post infection (FIG. 2B). This data demonstrates that JQ1 inhibits HCMV replication.

Upon the treatment of BET bromodomain inhibitor (+)-JQ-1, the viral progeny in the supernatant reduced dramatically. Without being limited by theory, this provides evidence that BET bromodomain inhibitors not only block the cell-mediated HCMV infection but also the release of viral particles.

### Example 3

This example demonstrates that the IC₅₀ of representative BET bromodomain inhibitor JQ1 against HCMV replication is lower than the dose used in anti-cancer experiments.

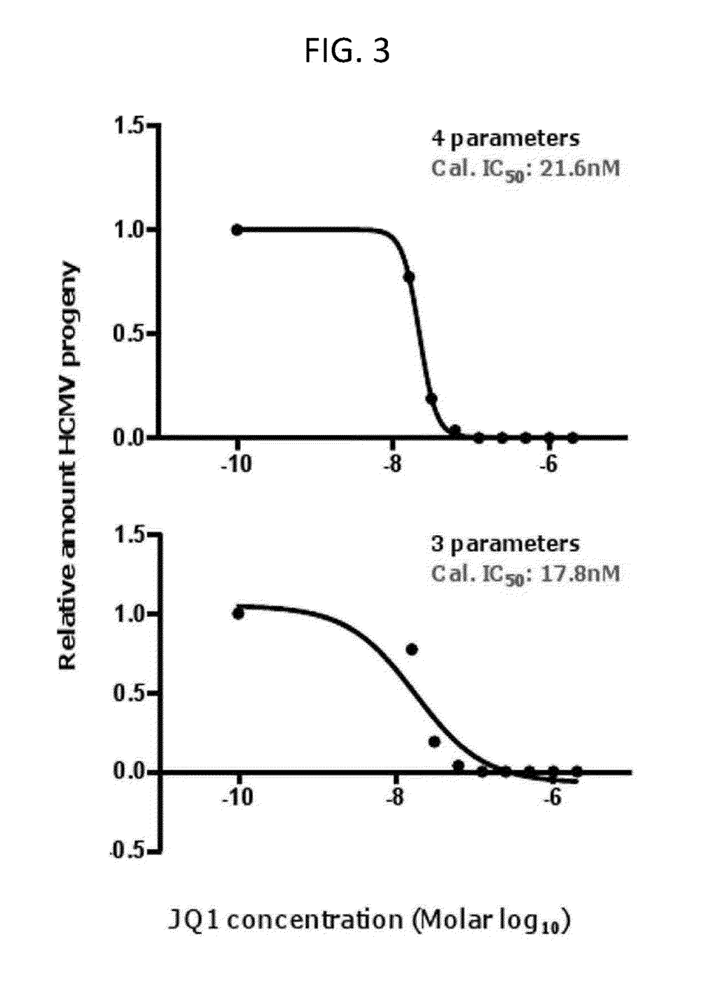

HFFs were infected with HCMV, strain AD 169, at an MOI of 3 in the presence of JQ1 at the range of 0-2000 nM. Culture media was changed every 24 hours to maintain the concentration of JQ1. At 5 days post infection, viral titers were determined by TCID₅₀. IC₅₀ (50% viral replication inhibitory concentration) was calculated from the dose response curve using Graphpad Prism 5 software. The calculated IC₅₀ of JQ1 using four parameters was 21.6 nM (FIG. 3A). The calculated IC₅₀ of JQ1 using three parameters was 17.8 nM (FIG. 3B). These calculated IC₅₀ values are much lower than published values used in the treatment of cancer.

The inventors used TCID₅₀ assays to quantify the IC₅₀ of (+)-JQ-1 in HCMV infection at a MOI of 3 (FIG. 3). The IC₅₀ is lower than the IC₅₀ determined by fluorescence reduction assays (Table 3). Without being limited by theory, this suggests that the release of productive viral particles might be more susceptible to BET bromodomain inhibitors than that of cell-to-cell mediated viral spread. Without being limited by theory, these experimental results provide a mode of action and advantages for the control of systemic viremia of HCMV-infected patients.

TABLE 3
Sensitivities of HCMV laboratory and clinical strains to bromodomain
inhibitors and current FDA-approved CMV antivirals

| | AD169-GFP | | TR-GFP | |
|---|---|---|---|---|
| | (Laboratory strain) | | (Clinical strain) | |
| | IC₅₀ (μM) | IC₉₀ (μM) | IC₅₀ (μM) | IC₉₀ (μM) |
| Bromodomain Inhibitors | | | | |
| (+)-JQ1 | 0.077 | 0.21 | 0.039 | 0.093 |
| (−)-JQ1* | 8.19 | 47.67 | 11.83 | 36.44 |
| RVX-208 | 7.93 | 31.1 | 3.16 | 6.67 |
| I-BET762 | 0.21 | 3.72 | 0.14 | 0.22 |
| (GSK525762A) | | | | |
| I-BET768 | 403.18 | N.D. | 13.4 | 24.55 |
| (GSK525768A)** | | | | |
| I-BET151 | 0.16 | 0.55 | 0.11 | 0.16 |
| (GSK1210151A) | | | | |
| PFI-1 | 0.79 | 2.56 | 0.42 | 1.218 |
| OTX-015 | 0.069 | 0.21 | 0.029 | 0.085 |
| CPI-203 | 0.04 | 0.14 | | |
| Bromosporine | 0.29 | 0.64 | | |
| FDA-approved CMV | | | | |
| antivirals | | | | |
| Ganciclovir | 1.78 | 24.96 | | |
| Cidofovir | 0.15 | 1.08 | | |
| Letermovir (AIC-246) | 0.0041 | 0.0058 | | |

*(−)-JQ1 is the stereoisomer of (+)-JQ1 and has no appreciable affinity to BET bromodomains.
**I-BET768 is the stereoisomer of I-BET762 and has no appreciable affinity to BET bromodomains.

### Example 4

This example demonstrates that JQ1 modestly inhibits the accumulation of HCMV late proteins even at high doses.

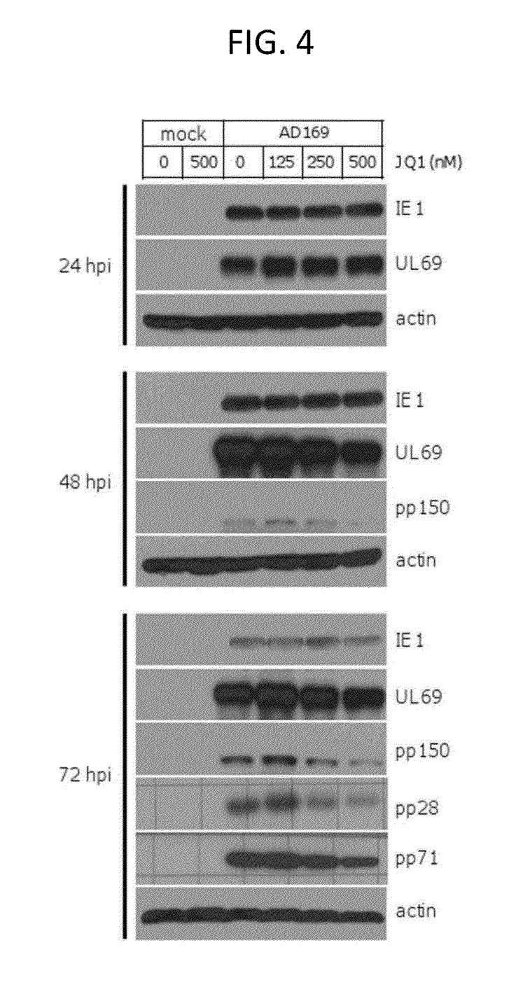

The method is as described by Perng et al. 2011. HFFs were infected with HCMV, strain AD169, at an MOI of 3 in the presence of different concentrations of JQ1. Culture media was changed every 24 hours to maintain the concentration of JQ1. Cells were harvested at 24, 48 and 72 hours post infection, HCMV proteins, immediate-early protein (IE1), early protein (UL69), and late proteins (pp71, pp150 and pp28) were determined by immunoblot analysis. (FIG. 4)

Without being limited by theory, the viral protein expression profiles (FIG. 4) provide evidence that inhibition of HCMV infection by BET bromodomain inhibitors is not majorly mediated by regulating viral gene expression. This inhibition is different than findings in studies of other herpesviruses such as EBV, a gamma-herpesvirus (Palermo et al., 2011). (CMV is a betaherpesvirus).

### Example 5

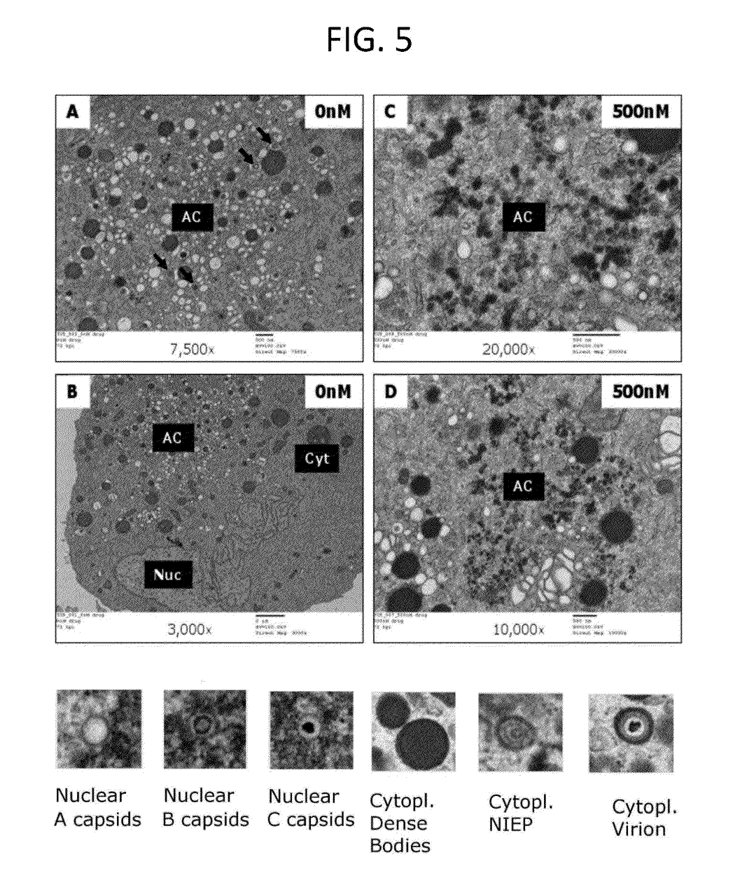

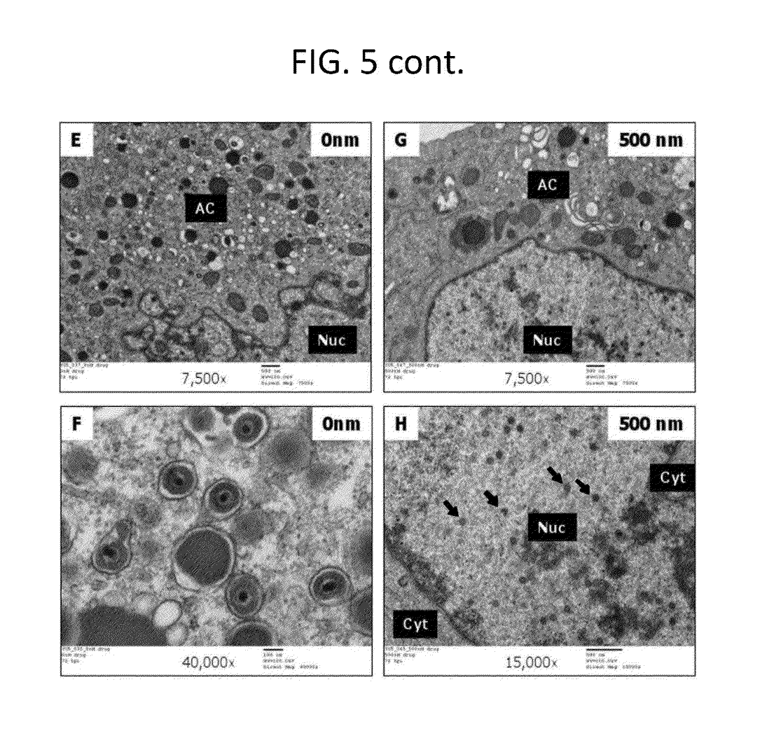

This example illustrates transmission electron micrographs of human cytomegalovirus (HCMV)-infected fibroblasts in the presence or absence of representative BET bromodomain inhibitor (+)-JQ-1. (FIG. 5).

In these experiments, HFFs were infected with AD169 strain at an MOI of 3 with or without JQ1 (500 nM). Culture media were changed every 24 hrs to maintain the concentration of JQ1. At 72 hpi, cells were harvested, fixed, and analyzed by transmission electronic microscopy.

The electron micrographs in FIG. 5 provide evidence that BET bromodomain inhibitor ((+)-JQ-1) blocks the production of infectious viral particles. The assembly compartments were not shown upon treatment. No capsid egressed from nucleus. Few capsids were seen in the nucleus but most of them are nuclear B capsids which do not contain viral DNA. Therefore, without being limited by theory, the major defect is likely at the step of forming DNA-containing (mature) capsids in the nuclei or capsid egress from the nucleus to the cytoplasm.

In the nucleus: A capsids lack scaffold as well as viral DNA and may result from abortive viral DNA encapsidation. B capsids contain scaffold but lack viral DNA. Without being limited by theory, they are likely to result from abortive capsid formation or DNA encapsidation. C capsids contain viral DNA and lack scaffold and they may represent nucleocapsids in the process of maturation.

In the cytoplasm: Dense bodies are noninfectious capsidless particles that carry pp65 tegument protein as the main constituent. Noninfectious enveloped particles (NIEP) can be produced when B capsids mature. Infectious virus particles (virions) can be produced when C capsids mature, containing encapsidated viral DNA.

### Example 6

This example illustrates that bromodomain inhibitors inhibit HCMV infection and spread.

HFF cells were infected with HCMV laboratory strain, AD169-GFP, at a MOI of 0.5. After virus adsorption, the virus inoculum was replaced with fresh medium containing respective BET bromodomain inhibitors followed by serial 2-fold dilutions. Culture media was changed every 24 hours to maintain the concentration of BET bromodomain inhibitors. Infected cells were examined by phase-contrast or fluorescence microscopy (Leica, Germany) at 10 days post infection (dpi).

FIG. 6 shows that treatments of BET bromodomain inhibitors block the spread of HCMV viral infection. The GFP-fluorescence images provide evidence that the BET bromodomain treatments reduced HCMV viral infection (indicated by the viral-expressed GFP). The bright field images provide evidence that the concentrations of BET bromodomain inhibitors in these experiments do not influence the viability of normal cells, even after 10-day treatment. This is inconsistent with previous literature reports regarding the studies of respective BET bromodomain inhibitors. The concentrations used in this experiment is similar or lower than those used for respective studies; I-BET151 (Dawson, M. A., et al. 2011), I-BET 762 (Dawson, M. A., et. al. 2011 and Nicodeme, E., et al. 2010), RVX-208 (Bailey, D., et al. 2010), PFI-1 (Picaud, S., et al. 2013).

### Example 7

This example illustrates representative in vitro dose-responsive curves of BET bromodomain inhibitors for HCMV laboratory and clinical strains.

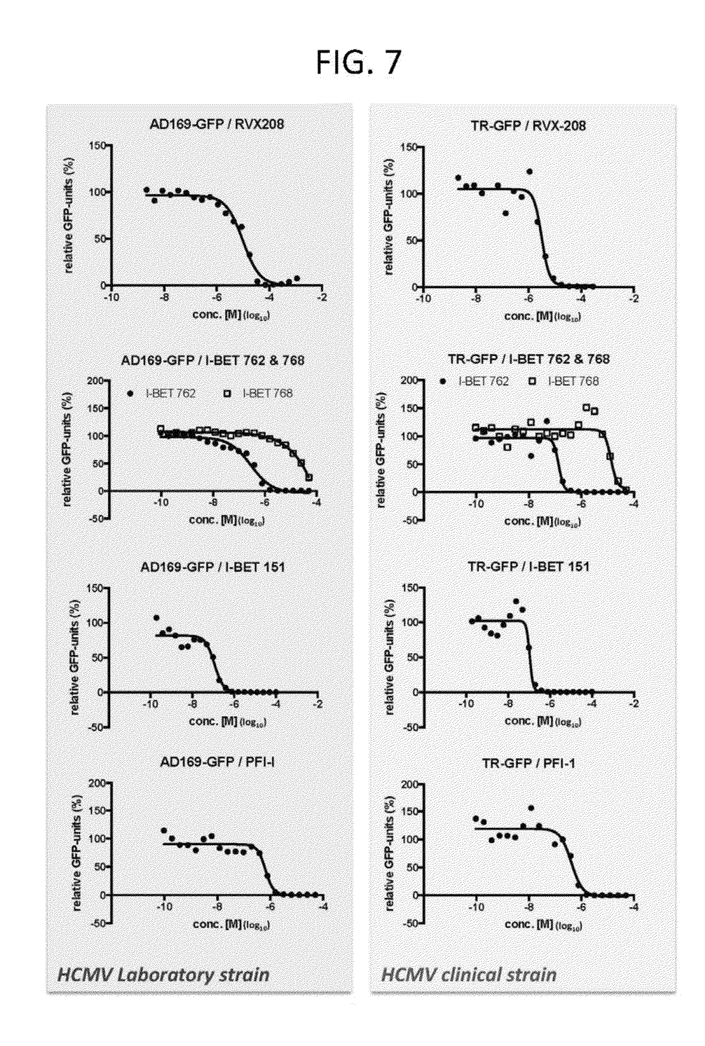

The dose-responsive curves of HCMV and clinical strains (FIG. 7) were determined by a GFP-based fluorescence reduction assay as described by Lischka, P., et al. 2010. For standard assays, HFF cells were cultured in black 96-well plates (Corning, USA) and infected with either recombinant laboratory-adapted strain AD169-GFP (MOI 0.3) or recombinant clinical strain TR-GFP (MOI 0.3). After virus adsorption, the virus inoculum was replaced with 200 μl medium containing the respective bromodomain inhibitors followed by serial 2-fold dilutions. Drug concentrations were tested at least in duplicate and the drug concentrations were maintained by replaced the medium every 24 hours. Plates were incubated at 37 C for 7-8 days. The medium was replaced by 200 μl PBS, and GFP units (GFPU) were determined by a fluorescence detector (BioTek Synergy H1, USA). Drug effects were calculated as a percentage of reduction in GFPU in the presence of each drug concentration comported to the GFPU determined in the absence of drug. The dose-response curves were calculated using the GraphPad Prism 6 (GraphPad Software, USA),

In this experiment, a stereoisomer of (+)-JQ-1, (−)-JQ-1 was used as a control. The inventors tested both laboratory strain (AD169-GFP) and clinical strain (TR-GFP). In both the laboratory strain and the clinical strain, the BET bromodomain inhibitor blocked HCMV infection as shown in FIG. 7.

### Example 8

This example illustrates representative in vitro dose-responsive curves of BET bromodomain inhibitors and current FDA-approved CMV antivirals.

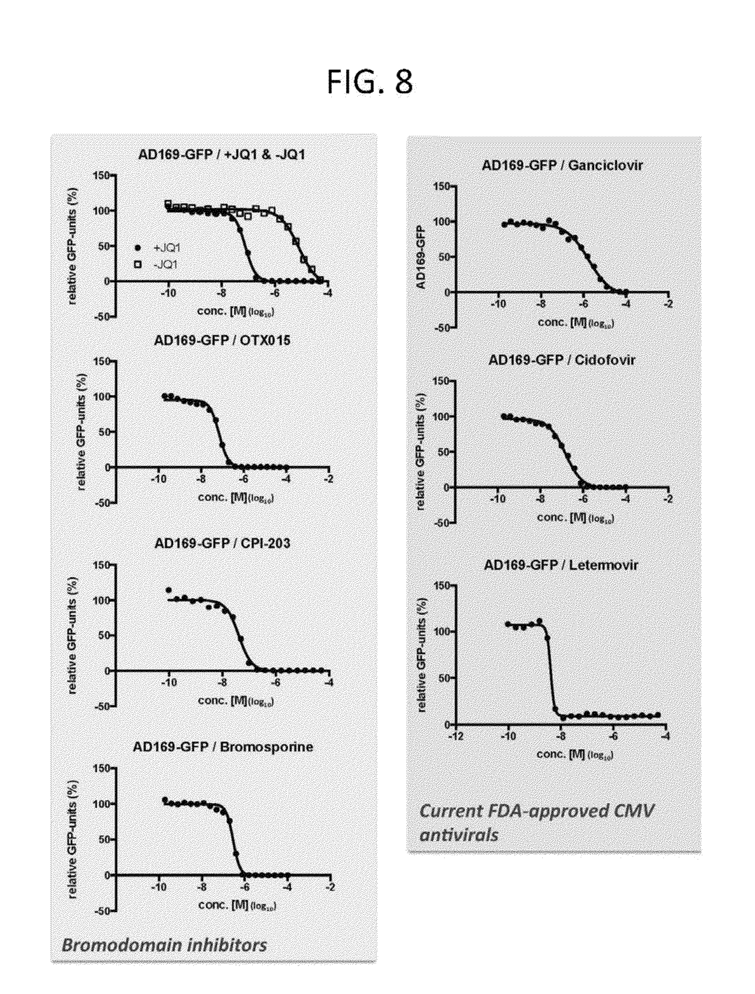

The dose-responsive curves of HCMV and current FDA-approved CMV antivirals (FIG. 8) were determined by a GFP-based fluorescence reduction assay as described by Lischka, P., et al. 2010. For standard assays, human foreskin fibroblast (HFF) cells were cultured in black 96-well plates (Corning, USA) and infected with recombinant laboratory-adapted strain AD169-GFP (MOI 0.3). After virus adsorption, the virus inoculum was replaced with 200 μl medium containing the respective bromodomain inhibitors or FDA-approved CMV antivirals followed by serial 2-fold dilutions. Drug concentrations were tested at least in duplicate and the drug concentrations were maintained by replaced the medium every 24 hours. Plates were incubated at 37 C for 7-8 days. The medium was replaced by 200 μl PBS, and GFP units (GFPU) were determined by a fluorescence detector (BioTek Synergy H1, USA), Drug effects were calculated as a percentage of reduction in GFPU in the presence of each drug concentration comported to the GFPU determined in the absence of drug, The dose-response curves were calculated using the GraphPad Prism 6 (GraphPad Software, USA).

In this experiment, we used stereoisomers of I-BET 762, I-BET 768, as a control. The inventors compared the dose-responsive curves of BET bromodomain inhibitors with current FDA approved/evaluating CMV antivirals. FIG. 8 illustrates a comparison of BET bromodomain inhibitors and CMV antivirals regarding concentration and dose-responses.

### Example 9

This example illustrates sensitivities of HCMV laboratory and clinical strains to BET bromodomain inhibitors and current FDA-approved CMV antivirals in fibroblast cells.

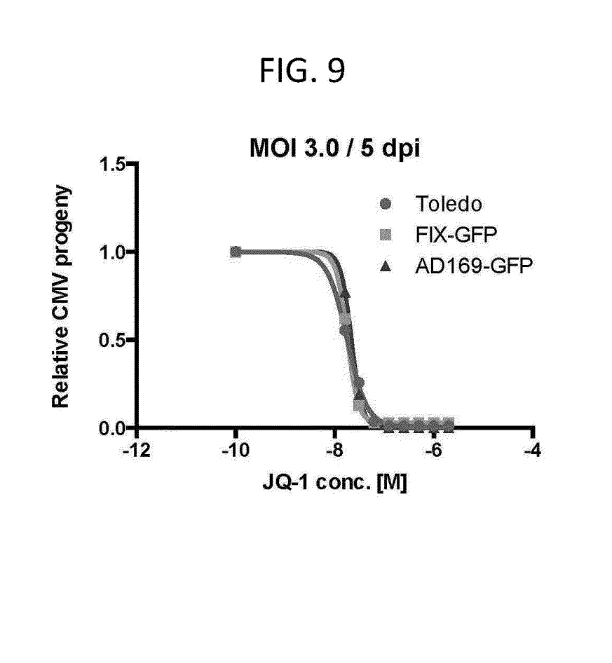

In these experiments, the inventors determined the IC₅₀ and IC₉₀ values of respective BET bromodomain inhibitors against HCMV infection using fluorescence reduction assay (FIG. 9; Table 3) s. The IC₅₀ and IC₉₀ values (drug concentrations producing 50% and 90% reduction in GFPU) were determined by a GFP-based fluorescence reduction assay as described by Lischka. P., et al. 2010. For standard assays, HFF cells were cultured in black 96-well plates (Corning, USA) and infected wish recombinant laboratory-adapted strain AD169-GFP (MOI 0.3) or TR-GFP (MOI 0.3). After virus adsorption, the virus inoculum was replaced with 200 μl medium containing the respective bromodomain inhibitors or FDA-approved CMV antivirals followed by serial 2-fold dilutions. Drug concentrations were tested at least in duplicate and the drug concentrations were maintained by replaced the medium every 24 hours. Plates were incubated at 37 C for 7-8 days. The medium was replaced by 200 μl PBS, and GFP units (GFPU) were determined by a fluorescence detector (BioTek Synergy H1, USA). IC₅₀ and IC₉₀ values were calculated using nonlinear regression curve fit with a variable slope (four parameters). GraphPad Prism 6 was used for the analysis.

The measured values are lower than those of these compounds in Bailey et al. 2010; Dawson et al. 2011; Filippakopoulos, P., et al. 2010; King et al. 2013; Nicodeme et al. 2010; Picaud et al. 2013; and Zuber et al. 2011.

### Example 10

This example illustrates MOI dependency of HCMV infection by treatment of representative BET bromodomain inhibitor (+)-JQ1.

IC₅₀ and IC₉₀ values (drug concentrations producing 50% and 90% reduction in GFPU) were determined by the fluorescence reduction assays (Table 4) as described by Lischka et al. 2010. For standard assays, human foreskin fibroblast (HFF) cells were cultured in black 96-well plates (Corning, USA) and infected with recombinant laboratory-adapted strains of AD169-GFP with various MOIs to compare MOI dependency of (+)-JQ-1 treatment. (MOIs of 1, 0.3, 0.1, and 0.03) After virus adsorption, the virus inoculum was replaced with 200 μl medium containing the respective bromodomain inhibitors followed by serial 2-fold dilutions. Drug concentrations were tested at least in duplicate and the drug concentrations were maintained by replaced the medium every 24 hours. Plates were incubated at 37 C for 7-8 days. The medium was replaced by 200 μl PBS, and GFP units (GFPU) were determined by a fluorescence detector (BioTek Synergy H1, USA). IC₅₀ and IC₉₀ values were calculated using nonlinear regression curve fit with a variable slope (four parameters). GraphPad Prism 6 was used for the analysis.

TABLE 4
MOI dependency of HCMV infection by treatment
of representative bromodomain inhibitor (+)-JQ1
AD169-GFP

| MOI | IC50 (μM) | IC90 (μM) |
|---|---|---|
| 1 | 0.0581 | 0.6016 |
| 0.3 | 0.0684 | 0.2227 |
| 0.1 | 0.059 | 0.1919 |
| 0.03 | 0.0586 | 0.1437 |

This experiment shows through the IC₅₀ results, that blocking of HCMV infection by the BET bromodomain inhibitor (+)-JQ-1 is less MOI dependent compared to known CMV antivirals. Since BET bromodomain inhibitors are less MOI dependent, BET bromodomain inhibitors may be used to treat severe HCMV viremia which currently requires high amounts of CMV antivirals to suppress infection with severe drug toxicity issues.

### Example 11

This example illustrates sensitivities of HCMV laboratory and clinical strains to BET bromodomain inhibitors determined by the release of viral particles (TCID₅₀ assay of culture supernatant).

In these experiments, the inventors used TCID₅₀ assays to quantify the IC₅₀ of (+)-JQ-1 in both HCMV laboratory-adapted and clinical strains (FIG. 9; Table 5). HFFs were infected with laboratory strain. AD169-GFP or laboratory strains FIXGFP & Toledo at an MOI of 3 in the presence of (+)-JQ-1 at the range of 0-2,000 nM. Culture media were changed every 24 hrs to maintain the concentration of JQ1. At 5 dpi, viral titers were determined by TCID₅₀. IC₅₀ (50% viral replication inhibitory concentration) was calculated from the dose response curve with the aid of Graphpad Prism 5 software.

Without being limited by theory, the low IC₅₀ values suggest that the release of productive viral particles is susceptible to BET bromodomain inhibitors independent of viral strains.

TABLE 5
Sensitivities of HCMV laboratory and clinical strains
to bromodomain inhibitors determined by the release
of viral particles (TCID₅₀ assay of supernatant)
(+)-JQ1

| | |
|---|---|
| IC₅₀ (μM) | IC₉₀ (μM) |

Laboratory strain

| | | |
|---|---|---|
| AD169-GFP | 0.018 | 0.049 |

Clinical strains

| | | |
|---|---|---|
| FIX-GFP | 0.018 | 0.031 |
| Toledo | 0.022 | 0.037 |

### Example 52

This example illustrates the effect of the time of addition of current CMV anti-virals (Ganciclovir, Letermovir, or Cidofovir) or representative BET bromodomain inhibitors ((+)-JQ1, I-BET 762, or OTX-015) on HCMV replication.

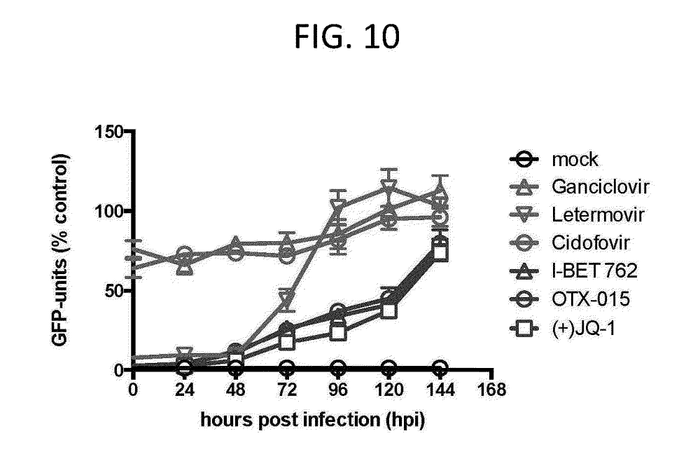

The method is as described by Lischka et al., 2010. HFF cells were infected with HCMV laboratory strain AD169-GFP and treated with fixed virus inhibitory concentration (˜6.5×IC50) of current FDA approved/evaluating CMV antivirals (Ganciclovir, Letermovir, cidofovir) or bromodomain inhibitors ((+)-JQ-1, IBET 762, OTX-015) at the indicated time points post-infection (hpi). After 7 days, cell supernatants were replaced by PBS and GFP units were determined. GFP units in compound-treated cells were compared to those in untreated cells, and the percentage of activity is plotted in FIG. 10. Results are averages for three experiments. Error bars indicate standard deviations.

The addition of drug assay shows that representatives bromodomain inhibitors ((+)-JQ-1/OTX-015/I-BET 762) block HCMV infections regardless of times post infection. (FIG. 10) The dosages required to control viral infections are low (6.5×IC₅₀ controlled viral infection efficiently). In contrast, current CMV antivirals (Ganciclovir, Cidofovir) require at least 10×IC₅₀ to control viral infection. Leterfovir can control viral infection when added before 48 hours post-infection, however, Leterfovir cannot control the viral infection after 48 hours post-infection. BET bromodomain inhibitors provide more flexibility for controlling viral infection.

### Example 13

This example illustrates transmission electron micrographs of HCMV clinical strain-infected fibroblasts in the presence or absence of representative BET bromodomain inhibitor (+)-JQ-1.

HFFs were infected with HCMV clinical strain TR-GFP at an MOI of 3 with or without (+)-JQ-1 (250 nM). Culture media were changed every 24 hrs to maintain the concentration of JQ1. At 72 hpi, cells were harvested, fixed, and analyzed by transmission electronic microscopy.

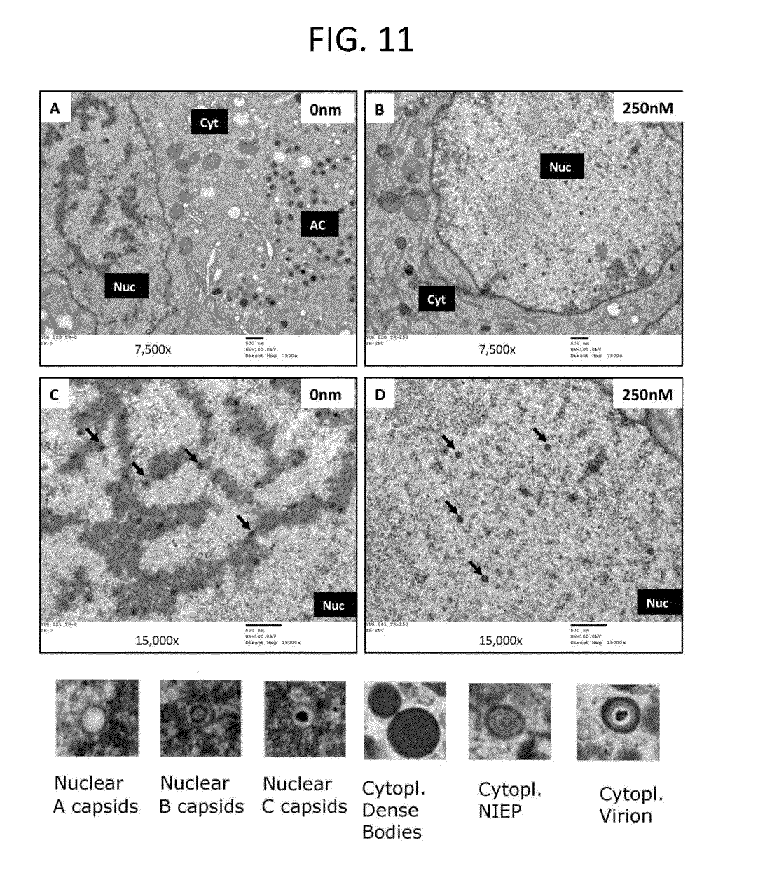

The EM analysis (FIG. 11) provides evidence that BET bromodomain inhibitor ((+)-JQ-1) blocks the production of infectious viral particles of HCMV, even the clinical strain. Low dosages of ((+)-JQ-1 were used (250 nM, ˜5-6.5 IC₅₀ depending on MOI). The phenotype displayed no capsid egressed from nucleus, few capsids seen in the nucleus but most of them are nuclear B capsids that do not contain viral DNA. Under this concentration, most of viral progeny production and cell-to-cell viral spread is inhibited (Table 3). However, based on the viral protein expression profile, the classes of viral proteins are expressed normally (FIG. 4). Without being limited by theory, the mode of action of BET bromodomain inhibitors against HCMV infection is mediated by something other than regulating viral gene expression.

### Example 14

This example illustrates that BET bromodomain inhibitor ((+)-JQ-1) inhibits the transcription of genes involved in glutamine uptake and metabolism induced by HCMV infection.

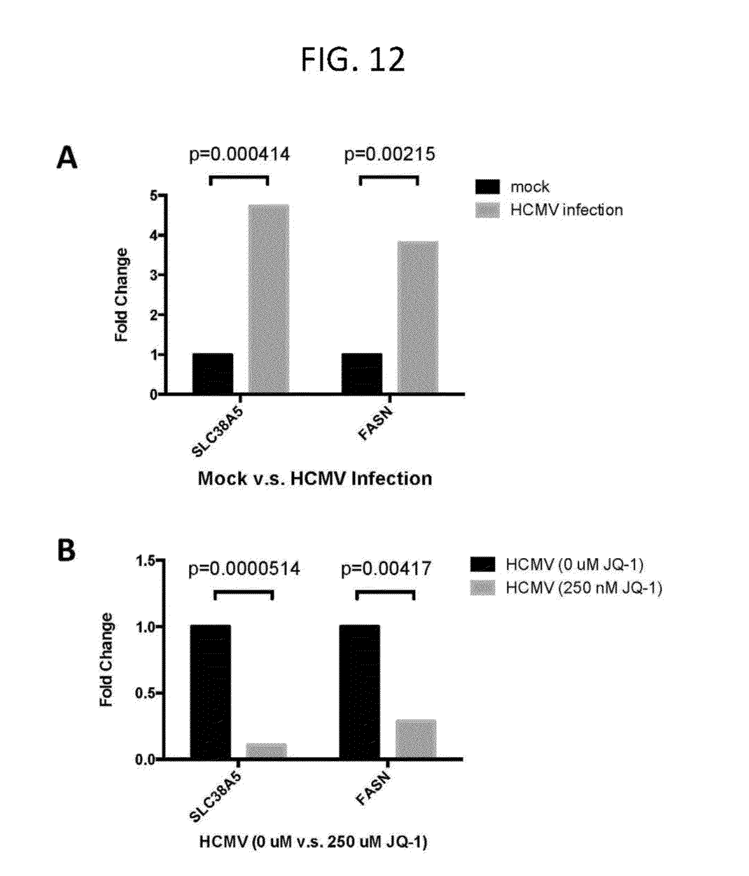

HFF cells were mock-infected or HCMV infected with laboratory strain AD169-GFP at a MOI of 3. (FIG. 12A) HFF cells were infected with AD169-GFP at a MOI of 3 in the presence or absence of 250 uM (+)-JQ-1. (FIG. 12B) Cells from both (A) and (B) were harvested at 48 hpi and the total RNA was extracted using a column-based RNA purification kit (Qiagen). RNA integrity was evaluated with a Nano-drop spectrometer (NanoDrop, Wilmington, Del.). Messenger RNA purification, fragmentation, construction of sequencing library and sequencing were performed. The differential expression profiles of two c-Myc inducible genes, fatty acid synthase (FASN) and solute carrier family 38 member 5 (SLC38A5), were determined using an EdgeR procedure.

FASN and SLC38A5 are two genes involved in lipogenesis and glucose/glutamine nutrient pathways. Both of them are induced by c-myc and shown to be up-regulated upon HCMV infection (Wise et al., 2008). The inventor's RNA-seq analysis shows that both genes are up-regulated by HCMV infection (FIG. 12A). However, the up-regulation is reversed by BET bromodomain inhibitor ((+)-JQ-1) (FIG. 12B). The lipogenesis and glutamine related metabolism pathways are blocked. Without being limited by theory, this is an explanation for why HCMV loses “cytomegaly” upon treatment (FIG. 1). The shortage of energy supply blocks the maturation of HCMV viral particle, even the viral protein expression is less affected (which is not less altered by lipogenesis/glutamineglu-related pathways).

BET bromodomain inhibitors are known to block downstream signaling of c-myc (Delmore et al., 2011). Blocking of lipogenesis or glutamine metabolism by targeting BET proteins/c-myc against viral infection is not previously known. Using BET bromodomain inhibitors to block c-myc and downstream lipogenesis/glucose-glutamine nutrient pathways for HCMV inhibition is not previously known.

KSHV, a DNA virus also belongs to Herpesvirus family, induces lipogenesis during latent viral infection (Delgado et al. 2012). However, during lytic infection, KSHV needs to suppress the lipogenesis master gene c-myc to facilitate actue/lytic infection (Lee et al. 2014).

BRD4 was reported as required to promote the transcription of certain EBV gene expression for its immortalization in B cells. Treatment of JQ-1 blocked the activity of certain gene promoters (Palermo et al., 2011). However, these genes are unique in EBV for its long-term latency/oncogenesis in B cells and not conserved among herpesviruses. Without being limited by theory, our examples showed that BET proteins play little roles in regulating HCMV gene expression (FIG. 4). Without being limited by theory, BET bromodomain inhibitors block HCMV infection by de-regulating the CMV-driven lipogenesis and metabolism pathways.

### Example 15

This example illustrates a method of inhibiting replication of human cytomegalovirus (HCMV) in a subject.

A patient is infected with HCMV. A health practitioner administers a therapeutically effective amount of the bromodomain inhibitor (+)-JQ1 by intraperitoneal injection. The patient's HCMV titers decrease.

### Example 16

This example illustrates a method of inhibiting replication of human cytomegalovirus (HCMV) in a subject.

A patient is infected with HCMV. A health practitioner administers an amount calculated to provide 19 μM of the bromodomain inhibitor RVX-208 by intraperitoneal injection. The patient's HCMV titers decrease.

### Example 17

This example illustrates a method of treating a human cytomegalovirus (HCMV) infection in a subject.

A patient is infected with HCMV. A health practitioner administers a therapeutically effective amount of the bromodomain inhibitor OTX-15 by oral administration. The patient's HCMV titers decrease.

### Example 18

This example illustrates a method of treating a human cytomegalovirus (HCMV) infection in a subject.

A patient is infected with HCMV. A health practitioner administers an amount calculated to provide 0.5 μM of the bromodomain inhibitor GSK1210151 by intraperitoneal injection. The patient's HCMV titers decrease.

### Example 19

This example illustrates the use of a bromodomain inhibitor for the treatment of human cytomegalovirus (HCMV) infection.

A patient is infected with HCMV. A health practitioner administers an amount calculated to provide 1 μM of the bromodomain inhibitor GSK525762A by intraperitoneal injection. The patient's HCMV titers decrease.

### Example 20

This example illustrates a method of inhibiting human cytomegalovirus (HCMV) replication in vitro.

A cell culture comprising a host cell infected with HCMV is provided. A laboratory technician contacts the host cell with an amount calculated to provide 1 μM of the bromodomain inhibitor PFI-1.

### Example 21

This example illustrates anti-HCMV activity of bromodomain inhibitors in cultured primary human fibroblasts. The concentrations to inhibit HCMV replication in these cells are reported in Table 6. No cell toxicity was observed at these effective concentrations.

TABLE 6
Sensitivities of HCMV in human fibroblasts
to bromodomain inhibitors

| Bromodomain | Concentration to inhibit |
|---|---|
| inhibitor | HCMV replication (μM) |
| PFI-1 | 1.391-0.781 |
| GSK525762 | 0.781-1.562 |
| RVX-208 | 14.063-28.125 |
| GSK1210151 | 0.391-0.781 |

These data illustrate that bromodomain inhibitors are able to inhibit HCMV replication without causing cell toxicity.

### REFERENCES

Anand, P., et al. BET bromodomains mediate transcriptional pause release in heart failure. Cell 2013 Aug. 1; 154(3): 569-82.

Arrowsmith, C. H., et al. Epigenetic protein families: a new frontier for drug discovery. Nat Rev Drug Discov. 2012 Apr. 13; 11(5): 384-400.

Bailey, D., et al. RVX-208: a small molecule that increases apolipoproiein A-I and high-density lipoprotein cholesterol in vitro and in vivo. J Am Coll Cardiol. 2010 Jun. 8; 55(23): 2580-9.

Banerjee, C., et al. BET bromodomain inhibition as a novel strategy for reactivation of HIV-1. J Leukoc Biol. 2012 December; 92(6): 1147-54.

Belkina, A. C., et al. BET domain co-regulators in obesity, inflammation and cancer. Nat Rev Cancer. 2012 Jun. 22; 12(7): 465-77.

Beroukhim, R., et al. The landscape of somatic copy-number alteration across human cancers. Nature. 2010 Feb. 18; 463(7283): 899-905.

Chambers, J. W., et al. Glutamine metabolism is essential for human cytomegalovirus infection. J Virol. 2010 February; 84(4): 1867-73.

Cheng, Z., et al. Inhibition of BET bromodomain targets genetically diverse glioblastoma. Clin Cancer Res. 2013 Apr. 1; 19(7): 1748-59.

Hagemeier, C., et al. The 72K IE1 and 80K IE2 proteins of human cytomegalovirus independently trans-activate the c-fos, c-myc and hsp70 promoters via basal promoter elements, J Gen Virol. 1992 September; 73 (Pt 9): 2385-93.

Dawson, M. A., et al. Inhibition of BET recruitment to chromatin as an effective treatment for MLL-fusion leukaemia. Nature. 2011 Oct. 2; 478(7370): 529-33.

Delgado, T., et al. Global metabolic profiling of infection by an oncogenic virus: KSHV induces and requires lipogenesis for survival of latent infection. PloS Pathog. 2012; 8(8): e1002866.

Delmore, J. E., et al. BET bromodomain inhibition as a therapeutic strategy to target c-Myc. Cell, 2011 Sep. 16; 146(6): 904-17.

Devaiah, B. N., et al. BRD4 is an atypical kinase that phosphorylates serine2 of the RNA polymerase II carboxy-terminal domain. Proc Natl Acad Sci USA. 2012 May 1; 109(18): 6927-32

Filippakopoulos, P., et al. Selective inhibition of BET bromodomains. Nature. 2010 Dec. 23; 468(7327): 1067-73

Goldner, T., et al. The novel anticytomegalovirus compound AIC246 (Letermovir) inhibits human cytomegalovirus replication through a specific antiviral mechanism that involves the viral terminase, J Virol. 2011 October; 85(20): 10884-93.

Hawkins, C., et al. Viruses and human brain tumors: cytomegalovirus enters the fray. J Clin Invest. 2011 October; 121(10): 3831-3.

Hebner, C. M., et al. Human papillomaviruses: basic mechanisms of pathogenesis and oncogenicity. Rev Med Virol. 2006 March-April; 6(2): 83-97.

King, B., et al. The ubiquitin ligase FBXW7 modulates leukemia-initiating cell activity by regulating MYC stability. Cell, 2013 Jun. 20; 153(7): 1552-66.

Lafemina, R. L., et al. Expression of the acidic nuclear immediate-early protein (IE1) of human cytomegalovirus in stable cell lines and its preferential association with metaphase chromosomes, Virology, 1989 October; 172(2): 584-600.

Lee, H. R., et al. Kaposi's sarcoma-associated herpesvirus viral interferon regulatory factor 4targets MDM2 to deregulate the p53 tumor suppressor pathway. J Virol. 2009 July; 83(3): 6739-47.

Lischka. P., et al. In vitro and in vivo activities of the novel anticytomegalovirus compound AIC246. Antimicrob Agents Chemother. 2010 March; 54(3): 1290-7.

Lockwood, W. W., et al. Sensitivity of human lung adenocarcinoma cell lines to targeted inhibition of BET epigenetic signaling proteins.

Loven, J., et al. Selective inhibition of tumor oncogenes by disruption of super-enhancers. Cell, 2013 Apr. 11; 153(2): 320-34. Proc Natl Acad Sci U S A, 2012 Nov. 20; 109(47): 19408-13.

Matzuk, M. M., et al. Small-molecule inhibition of BRDT for male contraception. Cell. 2012 Aug. 17; 150(4): 673-84.

Maxmen, A., et al. Cancer research: Open ambition. Nature. 2012 Aug. 9; 488(7410): 148-50.

McLure, K. G., et al. RVX-208, an inducer of ApoA-I in humans, is a BET bromodomain antagonist. PLoS One. 2013 Dec. 31; 8(12): e83190.

McPhillips, M. G., et al. Brd4 is required for e2-mediated transcriptional activation but not genome partitioning of all papillomaviruses. J Virol. 2006 October; 80(19): 9530-43.

McPhillips, M. G., et al. Interaction of bovine papillomavirus E2 protein with Brd4 stabilizes its association with chromatin. J Virol 2005 July; 79(14): 8920-32.

Mertz, J. A., et al. Targeting MYC dependence in cancer by inhibiting BET bromodomains. Proc Natl Acad Sci U S A. 2011 Oct. 4; 108(40): 16669-74.

Michael, M., et al. The story of human cytomegalovirus and cancer: increasing evidence and open questions. Neoplasia, 2009 January; 11(1): 1-9.

Monick, M. M., et al. The immediate early genes of human cytomegalovirus upregulate expression of the cellular genes myc and fos. Am J Respir Cell Mol Biol. 1992 September; 7(3): 251-6.

Mücke, K., et al. Human cytomegalovirus major immediate early 1 protein targets host chromosomes by docking to the acidic pocket on the nucleosome surface. J Virol. 2014 January; 88(2): 1228-48.

Nicodeme, E., et al. Suppression of inflammation by a synthetic histone mimic. Nature. 2010 Dec. 23; 468(7327): 1119-23.

Ott, C. J., et al. BET bromodomain inhibition targets both c-Myc and IL7R in high-risk acute lymphoblastic leukemia. Blood. 2012 Oct. 4; 120(14): 2843-52.

Ottinger, M., et al. Kaposi's sarcoma-associated herpesvirus LANA-1 interacts with the short variant of BRD4 and releases cells from a BRD4- and BRD2/RING3-induced G1 cell cycle arrest. J Virol. 2006 November; 80(21): 10772-86.

Palermo, R. D., et al. RNA polymerase II stalling promotes nucleosome occlusion and pTEFb recruitment to drive immortalization by Epstein-Barr virus. PLoS Pathog. 2011 October; 7(10): e1002334.

Perng, Y. C., et al. The human cytomegalovirus gene UL79 is required for the accumulation of late viral transcripts. J Virol. 2011 May; 85(10): 4841-52.

Picaud, S., et al. PFI-1, a highly selective protein interaction inhibitor, targeting BET Bromodomains. Cancer Res. 2013 Jun. 1; 73(11): 3336-46.

Platt, G. M., et al. Latent nuclear antigen of Kaposi's sarcoma-associated herpesvirus interacts with RING3, a homolog of the Drosophila female sterile homeotic (fsh) gene. J Virol. 1999 December; 73(12): 9789-95.

Puissant, A., et al. Targeting MYCN in neuroblastoma by BET bromodomain inhibition. Cancer Discov. 2013 March; 3(3): 308-23.

Rahbar, A., et al. Human cytomegalovirus infection levels in glioblastoma multiforme are of prognostic value for survival. J Clin Virol. 2013 May; 57(1): 36-42.

Reinhardt, J., et al. The carboxyl-terminal region of human cytomegalovirus IE1491aa contains an acidic domain that plays a regulatory role and a chromatin-tethering domain that is dispensable during viral replication. J Virol. 2005 January; 79(1): 225-33.

Sénéchal, H., et al. Amino acid substitutions that specifically impair the transcriptional activity of papillomavirus E2 affect binding to the long isoform of Brd4. Virology. 2007 Feb. 5; 358(1): 10-7.

Söderberg-Nauclér, C., et al. Survival in patients with glioblastoma receiving valganciclovir. N Engl J Med. 2013 Sep. 5; 369(10): 985-6.

Vastag, L., et al. Divergent effects of human cytomegalovirus and herpes simplex virus-I on cellular metabolism. PloS Pathog. 2011 July; 7(7): e1002124.

Viejo-Borbolla, A., et al. Brd2/RING3 interacts with a chromatin-binding domain in the Kaposi's Sarcoma-associated herpesvirus latency-associated nuclear antigen I (LANA-1) that is required for multiple functions of LANA-1. J Virol. 2005 November; 79(21): 13618-29.

Wilkinson, G. W., et al. Disruption of PML-associated nuclear bodies mediated by the human cytomegalovirus major immediate early gene product. J Gen Virol. 1998 May; 79 (Pt5): 1233-45.

Wise, D. R., et al. Myc regulates a transcriptional program that stimulates mitochondrial glutaminolysis and leads to glutamine addiction. Proc Natl Acad Sci U S A. 2008 Dec. 2; 105(48): 18782-7.

Wolmer-Solberg, N., et al. Frequent detection of human cytomegalovirus in neuroblastoma: a novel therapeutic target? Int J Cancer. 2013 Nov. 15; 133(10): 2351-61.

You, J., et al. Interaction of the bovine papillomavirus E2 protein with Brd4 tethers the viral DNA to host mitotic chromosomes. Cell. 2004 Apr. 30; 117(3): 349-60.

You, J., et al. Kaposi's sarcoma-associated herpesvirus latency-associated nuclear antigen interacts with bromodomain protein Brd4 on host mitotic chromosomes. J Virol. 2006 September; 80(18): 8909-19.

Yu, Y., et al. Viral effects on metabolism: changes in glucose and glutamine utilization during human cytomegalovirus infection. Trends Microbiol. 2011 July; 19(7): 360-7.

Yu, Y., et al. Human cytomegalovirus infection induces adipocyte-like lipogenesis through activation of sterol regulatory element binding protein 1. J Virol 2012 March; 86(6): 2942-9.

Zhang, F., et al. Dysregulated lipid metabolism in cancer. World J Biol Chem. 2012 Aug. 26; 3(8): 167-74.

Zhu, J., et al. Reactivation of latent HIV-1 by inhibition of BRD4. Cell Rep. 2012 Oct. 25; 2(4): 807-6.

Zuber, J., et al. RNAi screen identifies Brd4 as a therapeutic target in acute myeloid leukaemia. Nature. 2011 Aug. 3; 478(7370): 524-8.

All publications cited in this application are herein incorporated by reference in their entirety as if each individual publication, patent, patent application or other reference were specifically and individually indicated to be incorporated by reference.

What is claimed is:

1. A method of inhibiting replication of human cytomegalovirus (HCMV) in a subject, comprising administering a therapeutically effective amount of a bromodomain inhibitor to a subject in need thereof.

2. A method of inhibiting HCMV replication in accordance with claim 1, wherein the bromodomain inhibitor is (+)-JQ1.

3. A method of inhibiting HCMV replication in accordance with claim 1, wherein the bromodomain inhibitor is selected from the group consisting of PFI-1, GSK525762A, RVX-208, GSK1210151A and OTX-15.

4. A method of inhibiting HCMV replication in accordance with claim 1, wherein the bromodomain inhibitor is selected front the group consisting of a methyltriazolodiazepine-related compound, a 3,5-dimethylisoxazole-related compound, a 3-methyldihydroquinazolinone-related compound, a N-acetyl-2-methyltetrahydroquinoline-related compound, a quinazolone-related compound, a diazobenzene-related compound, triazolopyridazine-related compound, and a pyrrolopyridinone-related compound.

5. A method of inhibiting HCMV replication in accordance with claim 4, wherein the methyltriazolodiazepine-related compound is selected from the group consisting of CPI-203 ((S)-2-4-(4-chlorophenyl)-2,3,9-trimethyl-6H-thieno[3,2-f][1,2,4]triazolo[4,3-a][1,4diazepin-6-yl)acetamide), a 6-spiro-substituted triazolodiazepine, a dihydrobenzodiazepine, an isoxazoloazepine, a 6h-thieno[3,2-f][1,2,4]triazolo[4,3a][1,4]diazepine and MS-417 (methyl 2-(6S)-4-(4-chlorophenyl)-2,3,9-trimethyl-6H-thieno [3,2-f][1,2,4] triazolo[4,3-a][1,4]diazepin-6-yl)acetate).

6. A method of inhibiting HCMV replication in accordance with claim 4, wherein the N-acetyl-2-methyltetrahydroquinoline-related compound is 4-(2S, 4R)-{-1-acetyl-4-[(4-chlorophenyl) amino]-2-methyl-1,2,3,4-tetrahydro-6-quinolinyl}benzoic acid.

7. A method of inhibiting HCMV replication in accordance with claim 4, wherein the quinazolone-related compound is 2-[4-(2-hydroxyethoxy)-3,5-dimethyl-phenyl]-5,7-dimethoxy-3H -quinazolin-4-one.

8. A method of inhibiting HCMV replication in accordance with claim 4, wherein the triazolopyridazine-related compound is (S)-1-ethyl-3-(3-methyl-6-(methyl(1-phenylethyl)[1,2,4]triazolo[4,3-b]pyridazin-8-yl)urea

9. A method of inhibiting HCMV replication in accordance with claim 4, wherein the triazolopyridazine-related compound is bromosporine (N-[6-(3-methanesulfonamido-4-methylphenyl)-3-methyl-[1,2,4]triazolo[4,3-b]pyridazin-8-yl]carbamate.

10. A method of inhibiting HCMV replication in accordance with claim 4, wherein the pyrrolopyridinone-related compound is N-[4(2,4-difluorophenoxy)-3-(6-methyl-7-oxo-6,7-dihydro-1H-pyrrolo [2,3-c]pyridine-4-yl)phenyl]ethanesulfonamide.

11. A method of inhibiting HCMV replication in accordance with claim 5, wherein the 6-spiro-substituted triazolodiazepine is (1R,2R)-4′-4-Chlorophenyl)-N-ethyl-2′,3,9,-trimethylspiro-[cyclopropane-1,6′-thieno [3,2-f][1,2,4]triazolo[4,3-a][1,4]diazepine]-2-carboxamide.

12. A method of inhibiting HCMV replication in accordance with claim 5, wherein the dihydrobenzodiazepine is 4H-[1,2,4]triazolo[4,3-a][1,5]benzodiazepine,5,6-dihydro-1,4-dimethyl-8-(6-aminopyridin-3-yl)-6-(4-chloro-phenyl).

13. A method of treating a human cytomegalovirus (HCMV) infection in a subject, comprising administering a therapeutically effective amount of a bromodomain inhibitor to a subject is need thereof.

14. A method of treating human cytomegalovirus (HCMV) infection in accordance with claim 13, wherein the bromodomain inhibitor is (+)-JQ1.

15. A method of treating human cytomegalovirus (HCMV) infection in accordance with claim 13, wherein the bromodomain inhibitor is selected from the group consisting of PFI-1, GSK525762A, RVX-208, GSK1210151A, and OTX-15.

16. A method of treating human cytomegalovirus (HCMV) infection in accordance with claim 13, wherein the bromodomain inhibitor is selected from the group consisting of a methyltriazolodiazepine-related compound, a 3,5-dimethylisoxazole-related compound, a 3-methyldihydroquinazolinone-related compound, a N-acetyl-2-methyltetrahydroquinoline-related compound, a quinazolone-related compound, a diazobenzene-related compound, triazolopyridazine-related compound, and a pyrrolopyridinone-related compound.

17. A method of inhibiting human cytomegalovirus (HCMV) replication in vitro, comprising:
providing a culture comprising a host cell infected with HCMV; and
contacting the host cell with a bromodomain inhibitor.

18. A method in accordance with claim 17, wherein the bromodomain inhibitor is (+)-JQ1.

19. A method in accordance with claim 17, wherein the bromodomain inhibitor is selected from the group consisting of PFI-1, GSK525762A, RVX-208, GSK1210151A, and OTX-15.

20. A method in accordance with claim 17, wherein the bromodomain inhibitor is selected from the group consisting of a methyltriazolodiazepine-related compound, a 3,5-dimethylisoxazole-related compound, a 3-methyldihydroquinazolinone-related compound, a N-acetyl-2-methyltetrahydroquinoline-related compound, a quinazolone-related compound, a diazobenzene-related compound, triazolopyridazine-related compound, and a pyrrolopyridinone-related compound.
